(19) United States
(12) Patent Application Publication
(10) Pub. No.: US 2015/0250512 A1
(43) Pub. Date: Sep. 10, 2015

(54) Pedicle Screw Assembly

(71) Applicant: DEROYAL INDUSTRIES, INC., Powell, TN (US)

(72) Inventors: Nicholas Poker, Knoxville, TN (US); Travis Lane Turley, Knoxville, TN (US); Nephi Zufelt, Knoxville, TN (US)

(21) Appl. No.: 14/432,785

(22) PCT Filed: Oct. 15, 2013
(86) PCT No.: PCT/US13/65076

§ 371 Date: Apr. 1, 2015

Related U.S. Application Data
(60) Provisional application No. 61/715,620, filed on Oct. 18, 2012.

(51) Int. Cl. A61B 17/86

(52) U.S. Cl. CPC A61B 17/8605; A61B 17/866

(57) ABSTRACT

A pedicle screw assembly includes an elongated bone screw having an enlarged ball end. First and second seats are disposed on opposite sides of the ball end so that the ball is received between and seated within the first and second seats. An annular wave spring resiliently biases the first seat toward the second seat so as to provide a pre-clamping frictional engagement between the seats and the ball end to hold the seats in a selected position relative to the ball end.

### BACKGROUND ART

The present invention relates generally to a pedicle screw apparatus for providing an anchor point to a spinal segment for spinal fusion surgery.

### DISCLOSURE OF THE INVENTION

In one aspect the present invention provides a pedicle screw assembly including an elongated screw having an enlarged ball end. An upper housing part has an axial bore therethrough and has a lower counterbore. A downward facing annular surface joins the bore and counterbore. The upper housing part has two upward extending arms separated by a central slot for receiving an elongated member transversely through the slot. The slot has a bottom. A lower housing part has a lower opening through which the screw is received. The lower housing part has a lower seat located above the lower opening. The lower seat is shaped complementary to the ball end for receiving the ball end. A sleeve having a lower larger diameter end portion is received in the counterbore of the upper housing part. The sleeve also has an upper smaller diameter end portion received in the bore of the upper housing part. The sleeve has an upward facing annular shelf joining the larger and smaller diameter portions. The sleeve has an upper seat defined on a lower end of the sleeve. The upper seat is shaped complementary to the ball end for receiving the ball end. An annular wave spring is disposed concentrically about the upper smaller diameter end portion of the sleeve and is sandwiched between the downward facing annular surface of the upper housing part and the upward facing annular shelf of the sleeve. The sleeve is axially slidable relative to the upper housing part and the annular wave spring is dimensioned to provide a frictional pre-load on the ball end sandwiched between the upper and lower seats prior to the fixation of the elongated member in the upper housing part.

In another embodiment a pedicle screw assembly includes an elongated bone screw having an enlarged ball end. First and second seats are disposed on opposite sides of the ball end, so that the ball end is received between and seated within the first and second seats. An annular wave spring resiliently biases the first seat toward the second seat so as to provide a pre-clamping frictional engagement between the seats and the ball end to hold the seats in a selected position relative to the ball end. A housing contains the enlarged ball end and the first and second seats and the annular wave spring. The housing has longitudinally extending arms with a slot between the arms. The arms have internal threads. An externally threaded nut is engaged with the internal threads of the arms for simultaneously clamping a reinforcing rod in the slot and clamping the ball end in place between the first and second seats.

In another embodiment a method is provided for manufacturing a pedicle screw assembly. The method includes the steps of:

(a) assembling an elongated screw having a ball end between first and second seats shaped complementary to the ball end; and

(b) pre-loading the seats toward each other with an annular wave spring to provide a pre-clamping frictional engagement of the ball end with the seats, so that a housing containing the seats will remain in a selected position relative to the ball end prior to applying a final clamping force to the seats.

In any of the embodiments above the upper seat and/or the ball end may have scribe lines therein to increase friction between the seats and ball end.

In any of the embodiments above, the upper seat and/or the ball end may have a groove therein to increase friction between the seats and the ball end.

In any of the embodiments above the annular wave spring may comprise at least one and one-half complete coils.

In any of the embodiments above an upper nut may be threadedly engaged with an inner threaded surface of the arms of the upper housing part for fixation of the elongated member in the upper housing part.

In any of the embodiments above an upper end of the sleeve may extend above the bottom of the slot so that when the upper nut is securely engaged with the elongated member for fixation of the elongated member in the upper housing part, a clamping force is applied by the upper nut through the elongated member to the sleeve to also clamp the ball end between the upper and lower seats.

In any of the embodiments above the clamping force from the nut is not transmitted through the annular wave spring.

Numerous objects features and advantages of the present invention will be readily apparent to those skilled in the art upon a reading of the following disclosure when taken in conjunction with the accompanying drawings.

### BRIEF DESCRIPTION OF THE DRAWINGS

FIG. 1 is a schematic cross-section view showing two pedicle screw assemblies in place within a lumbar vertebra and showing two elongated rod members fixed in place within connector housings of the pedicle screw assemblies.

FIG. 2 is an exploded view of one of the pedicle screw assemblies.

FIG. 3 is a perspective view of an upper housing portion of the pedicle screw assembly.

FIG. 4 is a cross-sectional view of the upper housing portion of FIG. 3.

FIG. 5 is a perspective view of a sleeve of the pedicle screw assembly.

FIG. 6 is a cross-sectional view of the sleeve of FIG. 5.

FIG. 7 is a perspective view of a lower housing portion of the pedicle screw assembly.

FIG. 8 is a cross-sectional elevation view of the lower housing portion of FIG. 7.

FIG. 9 is a perspective view of an upper nut of the pedicle screw assembly.

FIG. 10 is a cross-section elevation view of the upper nut of FIG. 9.

FIG. 11 is a plan view of an annular wave spring of the pedicle screw assembly.

FIG. 12 is a schematic cross-section view of the wave spring of FIG. 11.

FIG. 13 is a perspective view of a driver apparatus for the pedicle screw assembly of FIG. 2, showing one of the pedicle screw assemblies in place on the distal end of the driver apparatus.

FIG. 14 is an elevation view of an upper driver housing portion.

FIG. 15 is an elevation partly sectioned view of a thumb wheel of the driver apparatus of FIG. 13.

FIG. 16 is an elevation view of a lower driver housing portion.

FIG. 17 is a cross-sectional elevation view of the lower driver housing portion of FIG. 16.

FIG. 18 is an elevation sectioned view of a tubular central driver housing portion.

FIG. 19 is an elevation sectioned view of an elongated spool handle of the driver apparatus.

FIG. 20 is an elevation view of a release shaft of the driver apparatus.

FIG. 21 is an elevation view showing the lower end of the driver apparatus about to engage with a pedicle screw assembly.

FIG. 22 is an elevation cross-sectioned view showing the lower end of the driver apparatus fully engaged with the pedicle screw assembly.

FIG. 23 is an elevation view showing the lower end of the driver apparatus connected to the pedicle screw assembly.

FIG. 24 is a right side elevation view of the apparatus of FIG. 23.

### BEST MODE FOR CARRYING OUT THE INVENTION

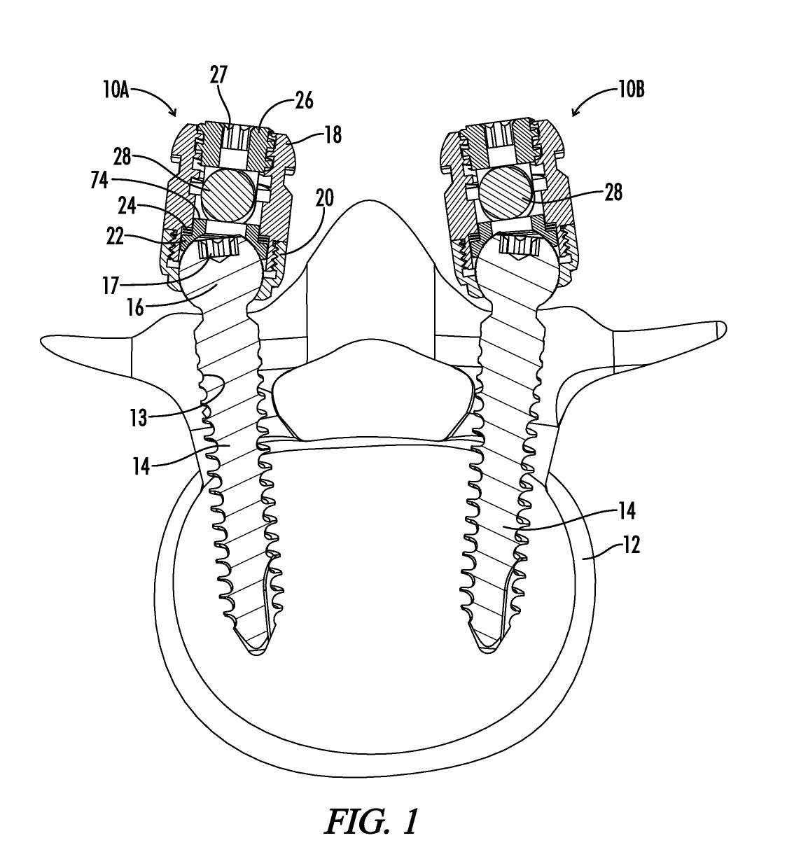

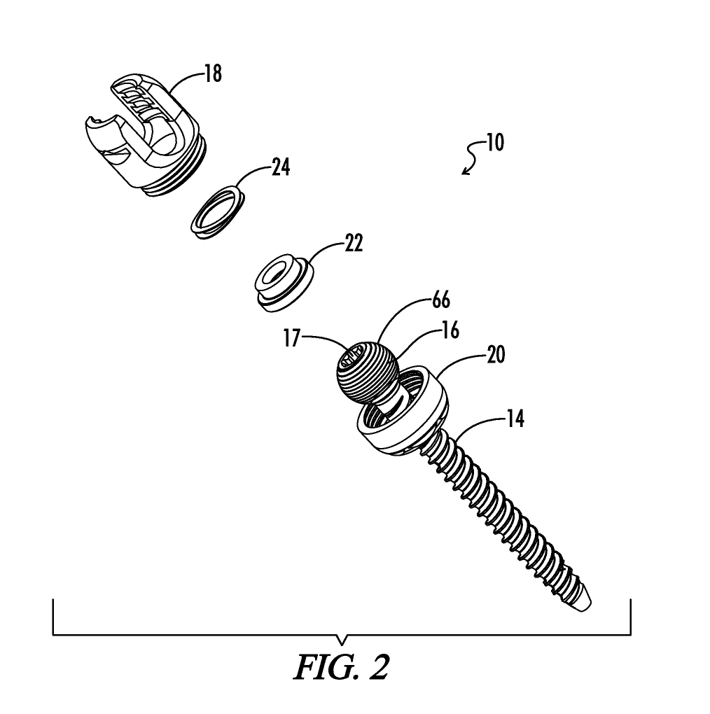

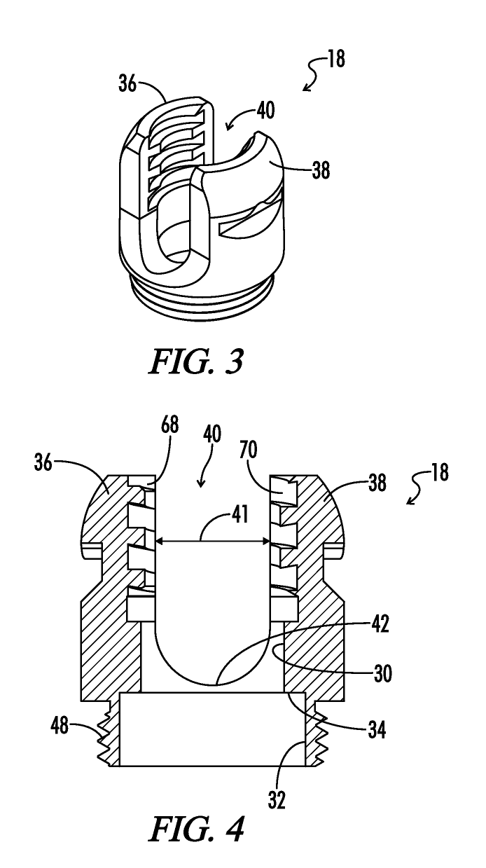

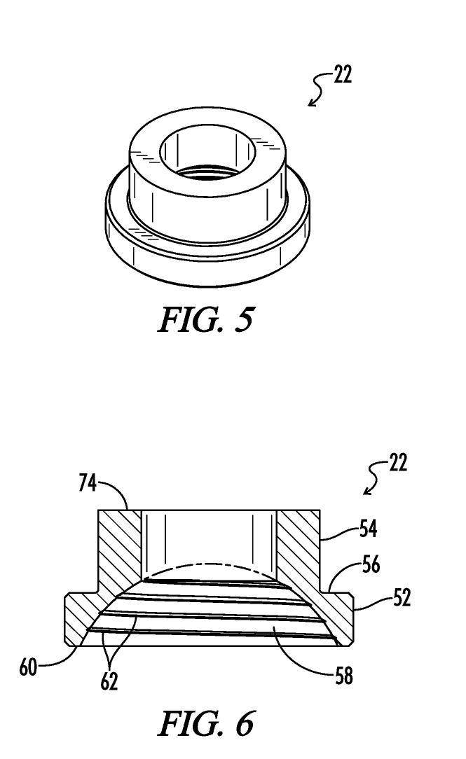

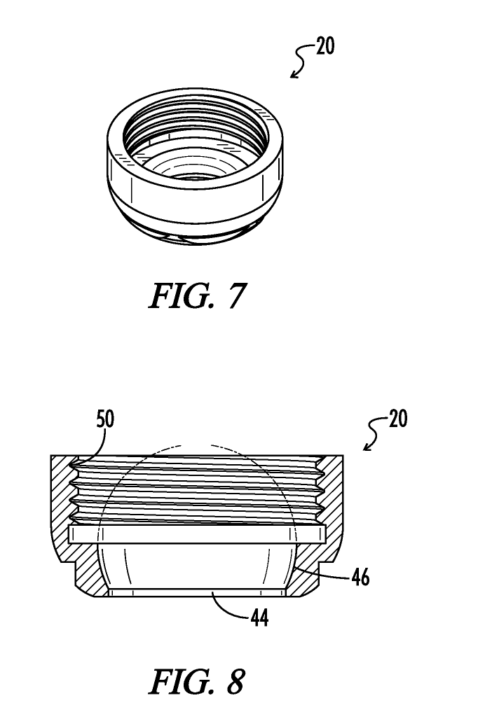

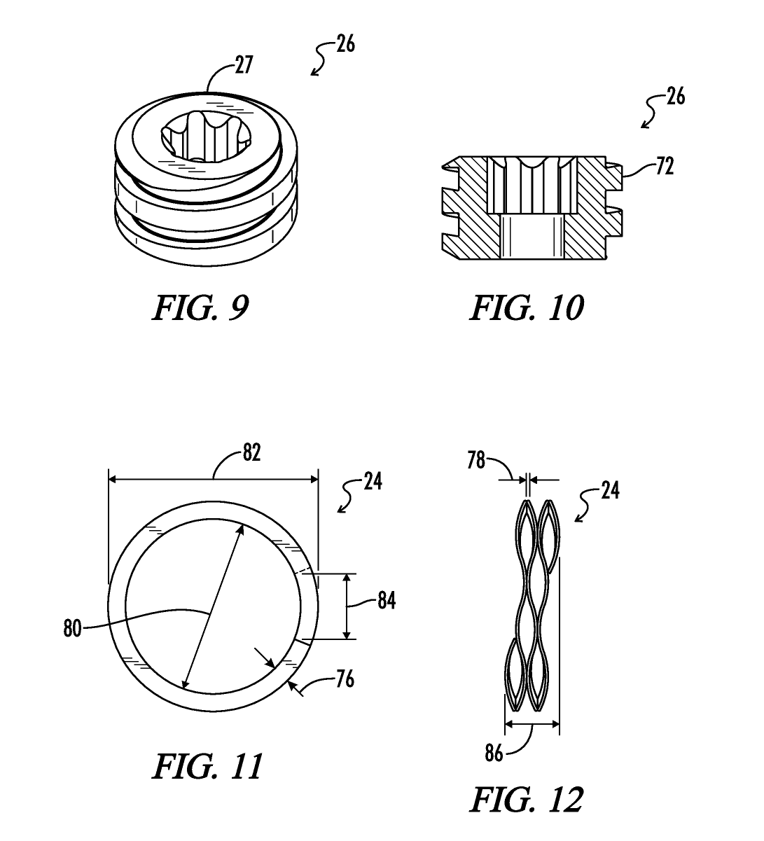

Referring now to FIGS. 1 and 2, a pedicle screw assembly is generally shown in exploded view in FIG. 2 and designated by the numeral 10. In FIG. 1 two of the pedicle screw assemblies are shown in place within a spinal segment 12, and the two pedicle screw assemblies are designated 10A and 10B.

Each of the pedicle screw assemblies 10 includes an elongated screw 14 having an enlarged ball end 16, an upper housing part 18 and a lower housing part 20, a sleeve 22, an annular wave spring 24, and an upper nut 26. The housing 18, 20 is sometimes referred to as a tulip housing or connector housing.

The enlarged ball end 16 has a multi-lobed socket 17 formed in the upper end thereof for engagement with a drive head of the driver apparatus 100 as further described below.

As seen in FIG. 1, each of the pedicle screw assemblies 10 is utilized to fix an elongated rod or elongated member 28 in place relative to the vertebra 12.

As best seen in FIG. 4, the upper housing portion 18, which may be also referred to as an upper housing part 18, has an axial bore 30 therethrough and has a lower counterbore 32. A downward facing annular surface 34 joins the bore 30 and the counterbore 32. The upper housing part 18 has two upward or longitudinally extending arms 36 and 38 separated by a central slot 40 for receiving the elongated rod 28 transversely through the slot 40. The slot 40 has a bottom 42.

The lower housing part 20 seen in FIG. 8 has a lower opening 44 through which the screw 14 is received. The lower housing part 20 has a lower seat 46 located above the lower opening 44. The lower seat 46 is shaped complementary to the ball end 16 for receiving the ball end 16 as seen in FIG. 1.

The lower end of upper housing part 18 has an external thread 48 which is received within an internal thread 50 of the upper end of lower housing part 20 so that the upper housing part 18 and the lower housing part 20 may be threadedly connected together by threads 48, 50.

The sleeve 22 has a lower larger diameter end portion 52 received in the counterbore 32 of the upper housing part 18. The sleeve 22 further has an upper smaller diameter end portion 54 which is received in the bore 30 of the upper housing part 18 as seen in FIG. 1. The sleeve 22 has an upward facing annular shelf 56 joining the larger and smaller diameter portions 52 and 54. The sleeve has an upper seat 58 defined on a lower end 60 of the sleeve 22. The upper seat 58 is shaped complementary to the ball end 16 for receiving the ball end 16 therein as seen in FIG. 1.

The annular wave spring 24 is disposed concentrically about the upper smaller diameter end portion 54 of the sleeve 22 and as seen in FIG. 1 is sandwiched between the downward facing annular surface 34 of the upper housing part 18 and the upward facing annular shelf 56 of the sleeve 22. The sleeve 22 is axially slidable relative to the upper housing part 38 and the annular wave spring 24 is dimensioned so as to provide a frictional pre-load on the ball end 16 sandwiched between the upper and lower seats 58 and 46 prior to fixation of the elongated rod 28 in the upper housing part 18.

The upper seat 58 may have scribe lines or grooves 62 therein. The ball end 16 may have scribe lines or grooves 66 defined therein. The use of the scribe lines or grooves 62 and 66, in any desired combination, increases friction between the seats and the ball end.

The upward extending arms 36 and 38 of the upper housing part 18 have internal threads 68 and 70 defined thereon. The upper nut 26 has a matching external thread 72 for receipt within the internal threads 68 and 70. The threads 68, 70 and 72 may for example be in the form of a single sided dove-tail threadform.

As best seen in FIG. 1, when the pedicle screws are in place and it is desired to fix the elongated rod 28 relative to the pedicle screw, the upper nut 26 is threadedly engaged with the threads 68 and 70 of the arms 36 and 38 to fix the elongated rod in the upper housing part 18. As can be seen in FIG. 1, an upper end 74 of the sleeve 22 extends above the bottom 42 of the slot 40 so that when the upper nut 26 is securely engaged with the elongated rod 28 for fixation of the elongated rod 28 in the upper housing part 18, a clamping force is applied by the upper nut through the elongated rod 28 to the sleeve 22 to also clamp the ball end 16 between the upper and lower seats 58 and 46. This clamping force is transmitted without passing through the annular wave spring 24.

The purpose of the annular wave spring 24 is to provide a pre-load on the sleeve 22 thus providing a pre-load sandwiching the ball end 16 between the upper and lower seats 58 and 46. This provides a frictional resistance of the housing assembly 18, 20 relative to the pedicle screw 14. The surgeon may rotate the housing assembly to a desired orientation relative to the pedicle screw 14 and the housing assembly will generally stay in place as the elongated rod 28 is placed therethrough. This allows the surgeon to assemble the spinal reinforcing structure made up of a plurality of the pedicle screw assemblies 10 and a plurality of the elongated rods 28 and associated cross-pieces (not shown).

The construction of the annular wave spring 24 is best seen in FIGS. 11 and 12. The wave spring illustrated is of the type generally referred to as a crest-to-crest wave spring, meaning that the crests of the waves as seen for example in FIG. 12 are joined together between adjacent coils of the wave spring. Other non crest-to-crest wave spring types may also be used. The spring for example may be constructed from a flat titanium wire having a width 76 and a thickness 78. The spring 24 may have an inside diameter 80 and an outside diameter 82, the difference in which is equal to the wire width 76.

In one embodiment, the spring 24 may comprise slightly less than two complete coils, with the coil ends being separated by a gap 84. This can be described as comprising at least 1- 1/2 complete coils. In one example, such a spring may be made from grade 2 titanium wire having a wire width 76 of 0.81 mm and a wire thickness 78 of 0.25 mm. The spring may have an outside diameter 82 slightly less than 9 mm in order to fit within a 9 mm bore. The wave pattern may provide 3- 1/2 waves for each 360° coil of the spring. The spring may have a free height 86 as seen in FIG. 12 of 1.30 mm. Other spring types having less than one and on-half coils may also be used. For example, a single coil wave spring may be used.

An annular wave spring 24 of the type just described provides a uniform application of force around the ball end 16 thus providing for smooth and reliable operation of the spring 24 to achieve its purpose of providing a reliable pre-load on the ball end 16 for holding the connector housing assembly in place relative to the ball end 16. This may be referred to as a pre-clamping frictional engagement between the seats and the ball end to hold the seats in a selected position relative to the ball end. Thus the annular wave spring is configured to apply a concentrically uniform pre-clamping load to the assembled first and second seats 58 and 46 and the ball end 16.

Methods of Manufacture of the Pedicle Screw Assembly

The pedicle screw assembly 10 is preferably pre-assembled by the manufacturer to include at least those components illustrated in the exploded view of FIG. 2. This assembly may be performed as follows.

The elongated screw 14 having the ball end 16 is placed through the open lower end 44 of lower housing part 20 and is engaged with lower seat 46. The spring 24 is placed about the upper smaller diameter portion 54 of sleeve 22, and the sleeve 22 is placed in engagement with the bore 30 and counterbore 32 of the upper housing part 18.

The upper and lower housing parts 18 and 20 are threadedly connected together by threads 48 and 50 thus assembling the elongated screw between the first and second seats, and pre-loading the seats toward each other with the annular wave spring 24 to provide a pre-clamping frictional engagement of the ball end 16 with the seats 58 and 46 so that the housing assembly 18, 20 containing the seats 58, 46 will remain in a selected position relative to the ball end 16 prior to applying a final clamping force to the seats.

The upper nut 26 is provided for later insertion within the upper housing part 18. The nut may or may not be pre-assembled with the upper housing part 18 by the manufacturer.

After the pedicle screw assemblies are put in place within the vertebra 12 as shown in FIG. 1, the elongated rods such as 28 are placed within the slots 40 of the upper housing parts 18. Then the nuts 26 are threadedly engaged with the internal threads of the upper housing to clamp the elongated rods 28 in place. This simultaneously clamps the ball end 16 between the upper and lower seats 58 and 46 to fix the position of the housing assembly relative to the ball end 16 and thus to fix the position of the elongated rods 28 relative to the pedicle screws 14.

The Driver Assembly

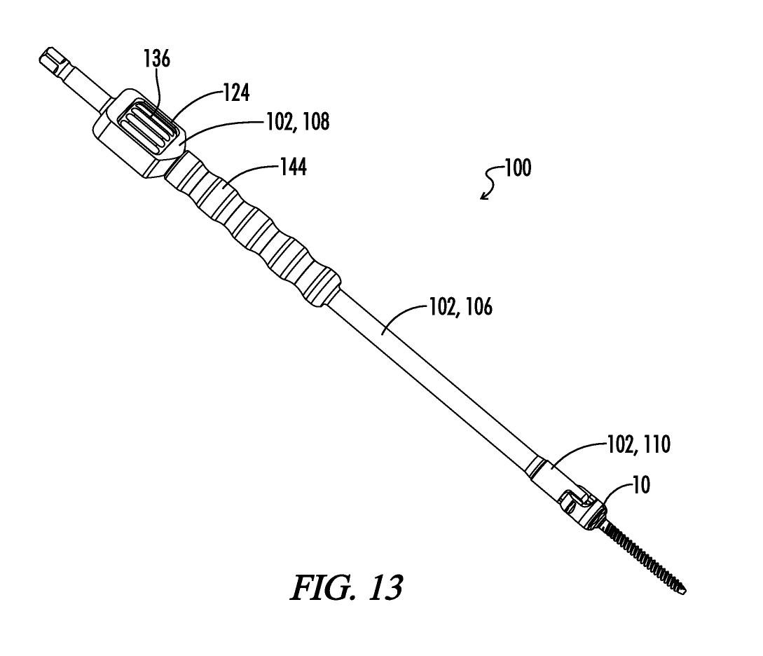

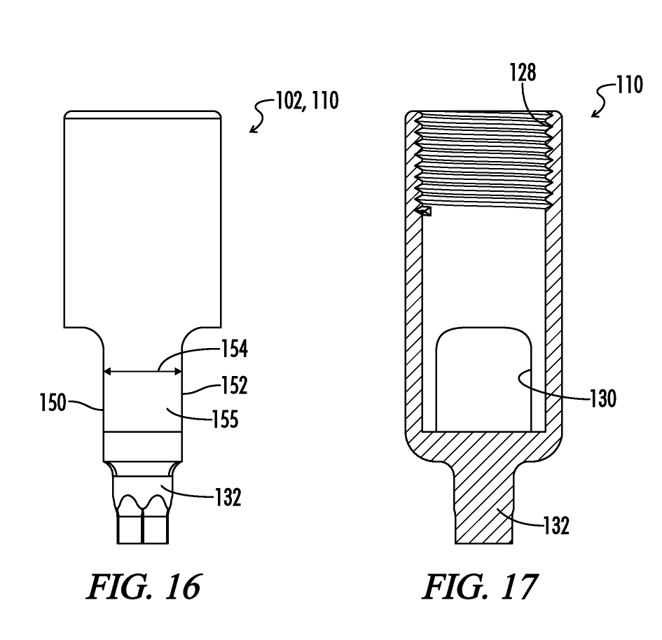

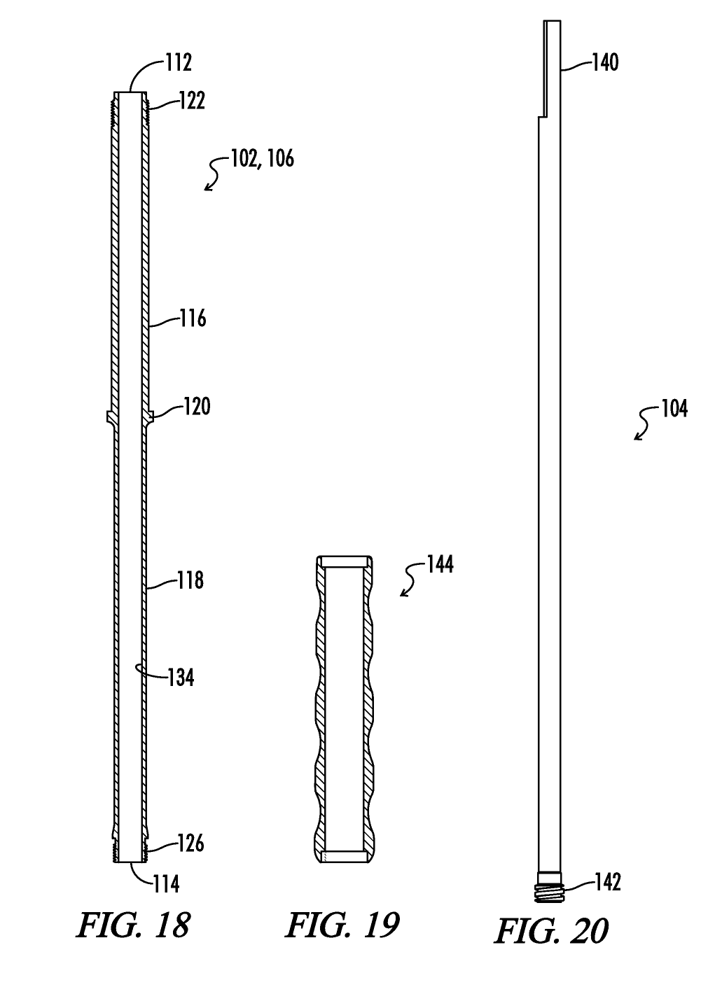

Referring now to FIGS. 13-24, a driver apparatus 100 for installing the pedicle screw assembly 10 is shown.

The driver apparatus 100 includes an elongated driver housing 102 and a release shaft 104 received in the driver housing 102.

The driver housing 102 is preferably a multi-part housing assembly including a tubular central driver housing portion 106, an upper driver housing portion 108, and a lower driver housing portion 110.

The central driver housing portion 106 includes upper and lower ends 112 and 114 which may also be referred to as proximal and distal ends 112 and 114.

The central driver housing portion 106 includes a larger diameter upper part 116 and a smaller diameter lower part 118 separated by an annular shoulder 120.

The upper driver housing portion 108 may also be referred to as an enlarged diameter knob housing portion 108, and is threadedly connected to the upper end 112 of central driver housing portion 106 by threads 122 and an internal thread (not shown) on the lower end of upper driver housing portion 108. The upper driver housing portion 108 has a first laterally open window 124 defined therethrough.

The lower driver housing portion 110, which may also be referred to as a distal housing portion 110, is attached to the lower end 114 of the central driver housing portion 106 by thread 126 on the central driver housing portion 106 and internal thread 128 on the lower driver housing portion 110. The lower driver housing portion 110 has a second laterally open window 130 defined therethrough.

The lower driver housing portion 110 has a screw drive head 132 defined thereon which has a multi-lobed shaped male driver configured to be received in a multi-lobed socket 17 defined in the upper end of the enlarged ball 16 of pedicle screw 14.

The central driver housing portion 106 has a longitudinal housing passage 134 defined therethrough which communicates with the upper laterally open window 124 and the lower laterally open window 130.

The release shaft 104 has a knob 136 attached to the upper end thereof. Knob 136 has a non-circular passage 138 which closely receives a non-circular upper portion 140 of the release shaft 104 so that the release shaft 104 and the knob 136 rotate together. As is best seen in FIG. 13, the knob 136 is received in and accessible through the upper laterally open window 124 defined in the upper housing portion 108.

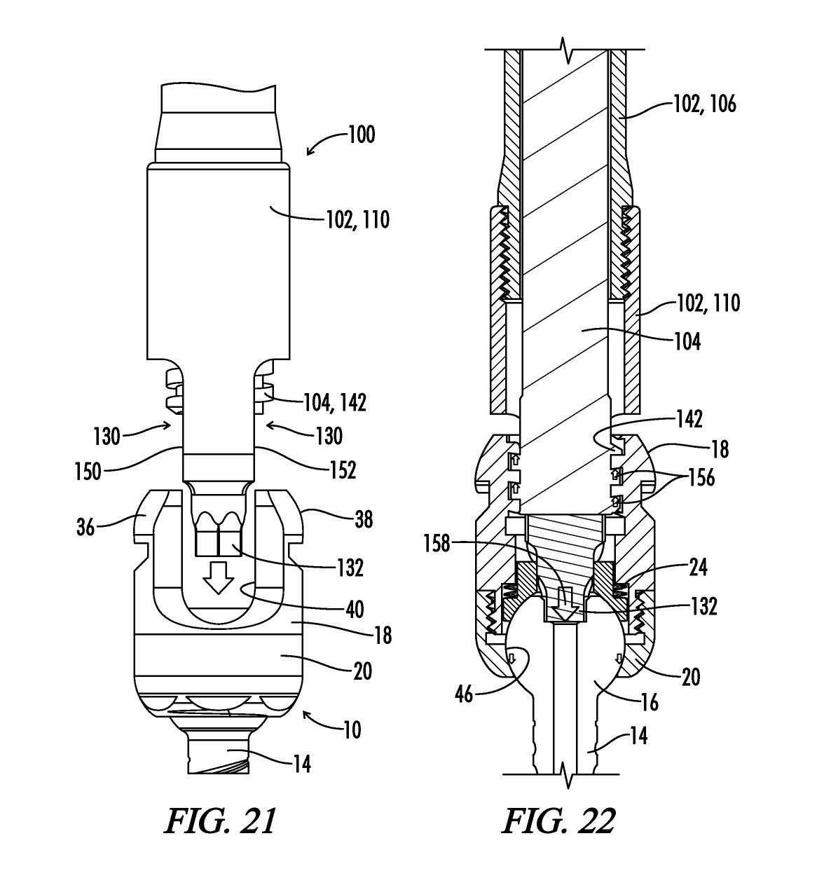

The release shaft 104 has a release thread 142 defined on a lower end portion of the shaft 104. The release thread 142 is received in and accessible through the lower laterally open window 130 as best seen in FIG. 21.

The release shaft 104 and knob 136 are rotatably received in the driver housing 102 so that the release thread 142 may be rotated by rotating the knob 136 while holding the driver housing 102 fixed.

An elongated spool handle 144 is best seen in FIG. 19 and is rotatably received about the larger diameter upper portion 116 of central driver housing portion 106.

The upper driver housing portion 108 has a drive shaft 146 extending upwardly therefrom, and the drive shaft 146 has an upper drive head 148 defined thereon which may be engaged by the surgeon with another drive tool to rotate the driver housing 102 while the surgeon steadies the driver housing 102 by grasping the spool handle 144. The driver housing 102 can rotate within the spool handle 144.

The lower driver housing portion 110 has first and second diametrically opposed parallel guide surfaces or guide surface portions 150 and 152 defined thereon on opposite sides of the lower laterally open window 130. The guide surface portions 150 and 152 are separated by a distance 154 which is less than the width 41 of slot 40 of the upper connector housing part 18 of the pedicle screw assembly 10. That portion of the lower driver housing 110 between the guide surfaces 150 and 152 may be referred to as a guide portion 155.

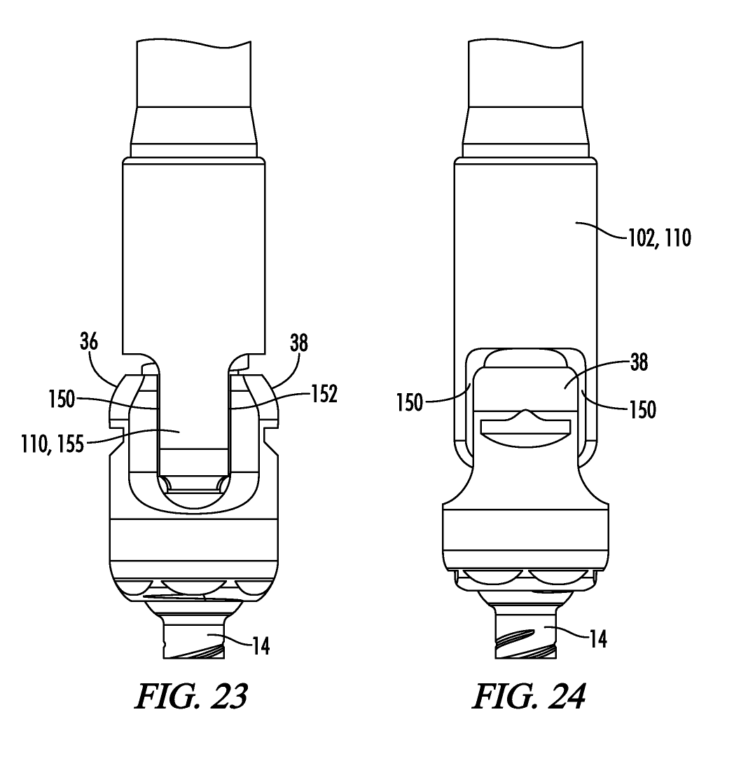

As seen in FIGS. 21 and 23, the guide surfaces 150 and 152 are configured to be closely received within the slot 40 of pedicle screw assembly 10 to hold the connector housing 18, 20 of the pedicle screw assembly 10 against rotation relative to the driver housing 102 when the release shaft 104 is rotated relative to the driver housing 102 to connect or disconnect the release thread 142 from the connector housing 18, 20 of the pedicle screw assembly 10.

As best seen in FIG. 22, the release thread 142 is configured to be threadedly engaged with the internal threads 68, 70 of the upper connector housing part 18 to connect or disconnect the driver apparatus 100 to or from the pedicle screw assembly 10 as is further described below.

Manner of Operation

Referring now to FIGS. 21-24, the manner in which the driver apparatus 100 may be utilized to install the pedicle screw assembly 10 is further described.

After the spinal vertebrae 12 have been prepared in a known manner for placement of the pedicle screws 14, the driver apparatus 100 may be connected to one of the pedicle screw assemblies 10 in the manner illustrated in FIGS. 21-24. First, as shown in FIG. 21, guide portion 155 of the lower driver housing 110, is inserted into the slot 40 of the connector housing 18, 20 between the upward extending arms 36 and 38. The screw drive head 132 is placed into the drive socket 17 in the upper end of the enlarged ball end 16 as shown in FIG. 22. The release shaft 104 is rotated relative to the driver housing 102 to begin to make up the release thread 142 with the internal threads 68, 70 of the upper connector housing portion 18. As the threaded engagement 142, 68, 70 is further made up, the driver apparatus 100 pulls the connector housing assembly 18, 20 and the pedicle screw 14 upward relative to the driver housing 102 until the screw drive head 132 is fully engaged with the socket 17 as generally shown in FIG. 22.

It is noted that when the release thread 142 is fully made up with the internal threads 68, 70 of upper connector housing 18 as generally shown in FIGS. 22-24, the release thread 142 is substantially circumferentially enclosed by the longitudinally overlapping lower portion of the lower housing 110 and the upward extending arms 36 and 38 of the upper connector housing 18.

It is noted that by substantially circumferentially enclosing the release thread 142 the release thread 142 is generally protected from contact with the patient's body tissues as the pedicle screw assembly 10 is placed by the driver apparatus 100 into the patient's body.

With reference to FIG. 22 it is noted that when the release threads 142 are fully made up with the threads 68, 70 of upper connector housing part 18 to fully engage the screw drive head 132 in the socket 17 of ball end 16 of pedicle screw 14, a tension load is placed across the connector housing 18, 20. This tension load is generally represented by the arrows 156 indicating an upward directed force being applied against the threads 68, 70 and by the arrow 158 indicating a downward force being applied to the ball end 16 and thus to the lower seat 46 of connector housing 18, 20. This tension force, plus the engagement of the screw drive head 132 with the socket 17 of the ball end 16 of pedicle screw 14 aids in holding the pedicle screw assembly 10 and particularly the pedicle screw 14 and connector housing 18, 20 in axial alignment with the driver apparatus 100 as seen in FIG. 22 so as to resist misalignment of the pedicle screw 14 and the connector housing 18, 20 during the application of driving torque to the pedicle screw 14 by the driver apparatus 100.

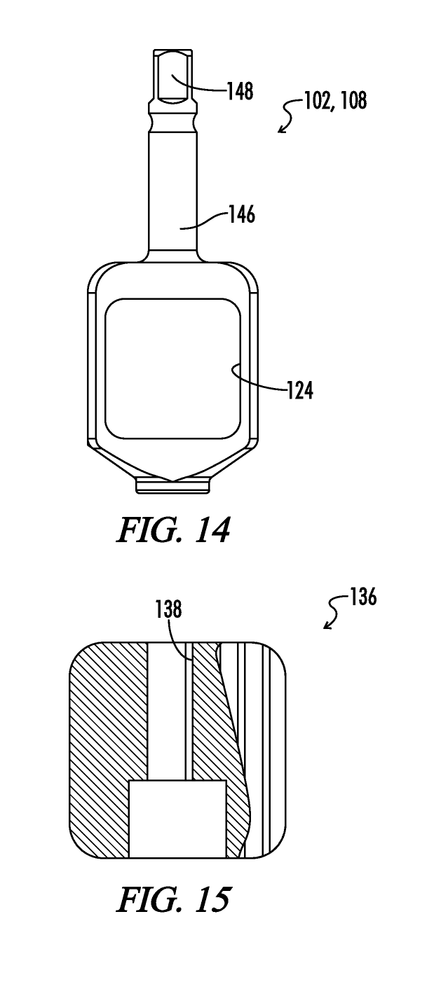

After the pedicle screw assembly 10 has been attached to the lower end of the driver apparatus 100 as generally shown in FIGS. 22-24, the pedicle screw 14 may be placed in the upper end of a prepared hole 13 of the spinal vertebra 12 and the surgeon may apply a driving torque to the pedicle screw 14 with the driver apparatus 100 thereby driving the pedicle screw 14 into position within the spinal vertebra 12 as generally shown in FIG. 1. This driving torque may be applied by rotating the driver housing 102 and particularly the screw drive head 132 defined on the lower end thereof while holding onto the spool handle 104. The driving torque may be applied to the driver housing 102 by placing a wrench or other tool on the upper drive head 148 seen in FIG. 14.

After the pedicle screw 14 has been driven into place within the spinal vertebra 12, the driver apparatus 100 may be released from the pedicle screw assembly 10 by unmaking the threaded engagement of the release thread 142 with the internal threads 68, 70 of the connector housing 18, 20. This is accomplished by holding the driver housing 102 fixed and rotating the release shaft and knob 136 counterclockwise relative to the driver housing 102 to unthread the release thread 102 from the connector housing 18, 20. The driver apparatus 100 is then withdrawn from the operating field.

At this point, the connector housing 18, 20 may be positioned by the surgeon in any desired rotational and angular relationship relative to the ball end 16, for example in positions like those shown in FIG. 1. The action of the annular wave spring 24 applies a pre-load between the upper and lower seats 58 and 46 across the ball end 16 so as to provide a frictional resistance to movement of the connector housing 18, 20 relative to the ball end 16.

Then, the elongated rods such as 28 may be placed in the slots 40 of a plurality of the pedicle screw assemblies 10, and then the elongated rods 28 may be clamped in place through the use of the upper nuts 26. The upper nuts 26 are placed in threaded engagement with the threads 68, 70 and are driven into place by another screwdriver (not shown) having a drive head which engages a socket 27 defined in the nut 26.

As the upper nut 26 is made up with the threads 68, 70 it bears down on the elongated rod 28 as seen in FIG. 1. The elongated rod 28 in turn bears down on the top of sleeve 22 which bears down via its seat 58 onto the spherical surface of ball end 16 which then bears down on the lower seat 46 to clamp the connector housing 18, 20 in place on the ball end 16 and to clamp the elongated rod 28 in place within the connector housing 18, 20 thus fixing the elongated rod 28 relative to the pedicle screw 14.

Thus it is seen that the apparatus and methods of the present invention readily achieve the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention as defined by the appended claims.

What is claimed is:

1. A pedicle screw assembly, comprising:
an elongated screw having an enlarged ball end;
an upper housing part having an axial bore therethrough and having a lower counterbore, and a downward facing annular surface joining the bore and the counterbore, the upper housing part having two upward extending arms separated by a central slot for receiving an elongated member transversely through the slot, the slot having a bottom;
a lower housing part having a lower opening through which the screw is received, the lower housing part having a lower seat located above the lower opening, the lower seat being shaped complementary to the ball end for receiving the ball end;
a sleeve having a lower larger diameter end portion received in the counterbore of the upper housing part, and an upper smaller diameter end portion received in the bore of the upper housing part, the sleeve having an upward facing annular shelf joining the larger and smaller diameter portions, the sleeve having an upper seat defined on a lower end of the sleeve, the upper seat being shaped complementary to the ball end for receiving the ball end; and
an annular wave spring disposed concentrically about the upper smaller diameter end portion of the sleeve and sandwiched between the downward facing annular surface of the upper housing part and the upward facing annular surface of the sleeve;
wherein the sleeve is axially slidable relative to the upper housing part and the annular wave spring is dimensioned to provide a frictional pre-load on the ball end sandwiched between the upper and lower seats prior to the fixation of the elongated member in the upper housing part.

2. The assembly of claim 1, wherein:
the upper seat and the ball end have scribe lines therein to increase friction between the seats and the ball end.

3. The assembly of claim 1, wherein:
the annular wave spring comprises at least one and one-half complete coils.

4. The assembly of claim 1, further comprising:
an upper nut threadedly engaged with an inner threaded surface of the arms of the upper housing part for fixation of the elongated member in the upper housing part.

5. The assembly of claim 4, wherein:
an upper end of the sleeve extends above the bottom of the slot so that when the upper nut is securely engaged with the elongated member for fixation of the elongated member in the upper housing part, a clamping force is applied by the upper nut through the elongated member to the sleeve to also clamp the ball end between the upper and lower seats.

6. The assembly of claim 5, wherein:
the clamping force is not transmitted through the annular wave spring.

7. A pedicle screw assembly, comprising:
an elongated bone screw having an enlarged ball end;
first and second seats disposed on opposite sides of the ball end, so that the ball end is received between and seated within the first and second seats;
an annular wave spring resiliently biasing the first seat toward the second seat so as to provide a pre-clamping frictional engagement between the seats and the ball end to hold the seats in a selected position relative to the ball end;
a housing containing the enlarged ball end and the first and second seats and the annular wave spring, the housing having longitudinally extending arms with a slot between the arms, the arms having internal threads; and
an externally threaded nut engaged with the internal threads of the arms for simultaneously clamping a reinforcing rod in the slot and clamping the ball end in place between the first and second seats.

8. The assembly of claim 7, wherein:
a clamping force load path from the nut to the seats does not pass through the annular wave spring.

9. The assembly of claim 7, wherein:
the annular wave spring is configured to apply a concentrically uniform pre-clamping load to the first seat.

10. The assembly of claim 7, wherein:
the second seat is defined internally on the housing.

11. The assembly of claim 10, further comprising:
a movable sleeve having the first seat defined on an end thereof; and
wherein the annular wave spring is concentrically disposed about the sleeve and engages an annular surface of the sleeve facing away from the first seat.

12. The assembly of claim 7, wherein:
the annular wave spring comprises at least one and one-half complete coils.

13. The assembly of claim 7, wherein:
the upper seat has a groove therein to increase friction between the upper seat and the ball end.

14. The assembly of claim 7, wherein:
the ball end has a groove therein to increase the friction between the seats and the ball end.

15. A method of manufacturing a pedicle screw assembly, comprising:
(a) assembling an elongated screw having a ball end between first and second seats shaped complementary to the ball end; and
(b) pre-loading the seats toward each other with an annular wave spring to provide a pre-clamping frictional engagement of the ball end with the seats, so that a housing containing the seats will remain in a selected position relative to the ball end prior to applying a final clamping force to the seats.

16. The method of claim 15, further comprising:
providing a clamping nut received within the housing and arranged to provide the final clamping force such that the final clamping force is not transmitted by the annular wave spring.

17. The method of claim 15, wherein:
step (b) further comprises placing the annular wave spring concentrically about a sleeve movable axially within the housing, the sleeve having the first seat defined thereon.

18. The method of claim 17, wherein:
in step (b) the housing is a two-piece housing having an upper housing part and a lower housing part, and step (b) further comprises:
inserting the elongated screw downward through a lower end opening of the lower housing part;
inserting the sleeve and the annular wave spring into the upper housing part; and
connecting the upper and lower housing parts to compress the annular wave spring.
